(19) United States
(12) Patent Application Publication
(10) Pub. No.: US 2022/0151468 A1
(43) Pub. Date: May 19, 2022

(54) DISTAL END COVER FOR ENDOSCOPE AND ENDOSCOPE APPARATUS

(71) Applicant: OLYMPUS CORPORATION, Tokyo (JP)

(72) Inventor: Koji YAMAYA, Tokyo (JP)

(73) Assignee: OLYMPUS CORPORATION, Tokyo (JP)

(21) Appl. No.: 17/592,613

(22) Filed: Feb. 4, 2022

Related U.S. Application Data
(63) Continuation of application No. PCT/JP2019/031505, filed on Aug. 8, 2019.

(51) Int. Cl. A61B 1/00; A61B 1/06

(52) U.S. Cl. CPC A61B 1/00101; A61B 1/00002; A61B 1/00091; A61B 1/0676

(57) ABSTRACT

A distal end cover for endoscope attached to and detached from a distal end portion of an insertion section of an endoscope includes a first cover including a nozzle for spraying fluid to at least an observation window or a part located in a visual field range of the observation window and a second cover including a flow channel for circulating, to the nozzle, the fluid fed from a fluid passage

### CROSS REFERENCE TO RELATED APPLICATION

This application is a continuation application of PCT/JP2019/031505 filed on Aug. 8, 2019, the entire contents of which are incorporated herein by this reference.

### BACKGROUND OF THE INVENTION

### 1. Field of the Invention

The present invention relates to a distal end cover for endoscope attached to and detached from a distal end portion of an insertion section of an endoscope, and an endoscope apparatus.

### 2. Description of the Related Art

Since an endoscope used in the medical field is inserted into a body cavity and used for the purpose of, in particular, testing and medical treatment, after the use, cleaning treatment, disinfection treatment, and sterilization treatment are necessary to reuse the endoscope.

However, since the cleaning treatment, the disinfection treatment, and the sterilization treatment for the endoscope take time, efficiency of use of the endoscope is deteriorated. Further, these kinds of treatment are very complicated.

Therefore, Japanese Patent Application Laid-Open Publication No. H7-47051 discloses a distal end cover for endoscope including a sheath that, when the distal end cover for endoscope is attached to an insertion section of an endoscope, specifically, fixed to a distal end portion of the insertion section, covers an outer surface of the insertion section in a longitudinal axial direction of the insertion section and an endoscope apparatus including the distal end cover for endoscope.

If the insertion section is inserted into a body cavity in a state in which the distal end cover for endoscope is attached, body fluid or the like in the body cavity does not adhere to the insertion section and adheres to only the distal end cover for endoscope including the sheath. Accordingly, after being removed from the body cavity, if the distal end cover for endoscope is detached from the insertion section and discarded, in reuse of the endoscope, it is possible to omit or simplify the various kinds of treatment described above for the endoscope after the use.

It is sometimes desired not to attach the distal end cover for endoscope to an insertion section of one endoscope and to use the endoscope alone.

Further, it is also conceivable to use the distal end cover for endoscope in an insertion section of an endoscope usable alone.

In the distal end cover for endoscope and the endoscope apparatus, there has been a demand for a configuration that can properly use, for the insertion section of the one endoscope, a use form for covering the insertion section with the sheath and a use form for not covering the insertion section with the sheath and can surely remove contaminants in a visual field range of an observation window.

### SUMMARY OF THE INVENTION

In order to achieve the object described above, a distal end cover for endoscope according to an aspect of the present invention is a distal end cover for endoscope attached to and detached from a distal end portion of an insertion section of an endoscope, the distal end cover for endoscope including: a first cover including a nozzle for spraying fluid to at least an observation window provided at the distal end portion of the insertion section or a part located in a visual field range of the observation window; and a second cover, at least a part of which engages with a circumferential surface of the first cover, the second cover including a flow channel for circulating, to the nozzle, the fluid fed from a fluid passage of the endoscope.

An endoscope apparatus according to an aspect of the present invention includes: an endoscope including an insertion section inserted into a subject; a distal end cover for endoscope attached to and detached from a distal end portion of the insertion section, the distal end cover for endoscope including a first cover including a nozzle for spraying fluid to at least an observation window provided at the distal end portion of the insertion section or a part located in a visual field range of the observation window and a second cover, at least a part of which engages with a circumferential surface of the first cover, the second cover including a flow channel for circulating, to the nozzle, the fluid fed from a fluid passage of the endoscope; a sheath water-tightly attached to an outer circumferential surface or an inner circumferential surface of the first cover or the second cover and covering an outer surface of the insertion section of the endoscope in a longitudinal axial direction of the insertion section; and a light transmitting plate having light transmissivity including a part water-tightly attached to the first cover or the second cover and located in the visual field range of the observation window and including a first surface opposed to the observation window of the insertion section to which the distal end cover for endoscope is attached and a second surface located on an opposite side of the first surface, the fluid being sprayed to the second surface from the nozzle.

### BRIEF DESCRIPTION OF THE DRAWINGS

FIG. 1 is a diagram schematically showing a configuration of an endoscope apparatus to which a distal end cover for endoscope in a first embodiment is attached;

FIG. 2 is a partially enlarged perspective view showing a state in which a distal end cover not including a sheath is attached to a distal end portion of an insertion section of an endoscope shown in FIG. 1;

FIG. 3 is an exploded perspective view showing a state in which the distal end cover is detached from the distal end portion shown in FIG. 2;

FIG. 3A is a front view of the distal end cover shown in FIG. 3;

FIG. 3B is a top view of the distal end cover shown in FIG. 3;

FIG. 3C is a bottom view of the distal end cover shown in FIG. 3;

FIG. 3D is a side view of the distal end cover shown in FIG. 3;

FIG. 3E is another side view of the distal end cover shown in FIG. 3;

FIG. 3F is a rear view of the distal end cover shown in FIG. 3;

FIG. 4 is an exploded perspective view of the distal end cover shown in FIG. 3;

FIG. 4A is a front view of a first cover in the distal end cover shown in FIG. 4;

FIG. 4B is a top view of the first cover in the distal end cover shown in FIG. 4;

FIG. 4C is a bottom view of the first cover in the distal end cover shown in FIG. 4;

FIG. 4D is a side view of the first cover in the distal end cover shown in FIG. 4;

FIG. 4E is another side view of the first cover in the distal end cover shown in FIG. 4;

FIG. 4F is a rear view of the first cover in the distal end cover shown in FIG. 4;

FIG. 4G is a front view of a second cover in the distal end cover shown in FIG. 4;

FIG. 4H is a top view of the second cover in the distal end cover shown in FIG. 4;

FIG. 4I is a bottom view of the second cover in the distal end cover shown in FIG. 4;

FIG. 4J is a side view of the second cover in the distal end cover shown in FIG. 4;

FIG. 4K is another side view of the second cover in the distal end cover shown in FIG. 4;

FIG. 4L is a rear view of the second cover in the distal end cover shown in FIG. 4;

FIG. 5 is a front view of the distal end portion and the distal end cover shown in FIG. 2 viewed in a V direction in FIG. 2;

FIG. 6 is a partial cross-sectional view of the distal end portion and the distal end cover taken along a VI-VI line in FIG. 5;

FIG. 7 is a partial cross-sectional view showing a state in which the distal end cover is detached from the distal end portion shown in FIG. 6;

FIG. 8 is a partially enlarged perspective view showing a state in which a distal end cover including a sheath is attached to the distal end portion of the insertion section of the endoscope shown in FIG. 1;

FIG. 9 is an exploded perspective view showing a state in which the distal end cover is detached from the distal end portion shown in FIG. 8;

FIG. 10 is an exploded perspective view of the distal end cover shown in FIG. 9;

FIG. 11 is a front view of the distal end portion and the distal end cover shown in FIG. 8 viewed in a XI direction in FIG. 8;

FIG. 12 is a partial cross-sectional view of the distal end portion and the distal end cover taken along a line in FIG. 11;

FIG. 13 is a partial cross-sectional view showing a state in which the distal end cover is detached from the distal end portion shown in FIG. 12;

FIG. 14 is a diagram showing a state in which a plug is attached to a treatment instrument insertion pipe sleeve and a suction pump is connected to a suction pipe sleeve in the state in which the distal end cover including the sheath is attached to the distal end portion of the insertion section of the endoscope shown in FIG. 1;

FIG. 15 is a diagram showing a state in which a suction switching button shown in FIG. 14 is closed and the sheath adheres through suction via a channel by the suction pump;

FIG. 16 is a partially enlarged perspective view showing a state in which a distal end cover not including a sheath and including a light transmitting plate is attached to the distal end portion of the insertion section of the endoscope shown in FIG. 1;

FIG. 17 is an exploded perspective view showing a state in which the distal end cover is detached from the distal end portion shown in FIG. 16;

FIG. 18 is an exploded perspective view of the distal end cover shown in FIG. 17;

FIG. 19 is a perspective view showing a modification in which a channel is externally attached to a first cover in the distal end cover shown in FIG. 8;

FIG. 20 is a partially enlarged perspective view showing a state in which a distal end cover not including a sheath is attached to a distal end portion of an insertion section of an endoscope in an endoscope apparatus in a second embodiment;

FIG. 21 is an exploded perspective view showing a state in which the distal end cover is detached from the distal end portion shown in FIG. 20;

FIG. 21A is a front view of the distal end cover shown in FIG. 21;

FIG. 21B is a top view of the distal end cover shown in FIG. 21;

FIG. 21C is a bottom view of the distal end cover shown in FIG. 21;

FIG. 21D is a side view of the distal end cover shown in FIG. 21;

FIG. 21E is another side view of the distal end cover shown in FIG. 21;

FIG. 21F is a rear view of the distal end cover shown in FIG. 21;

FIG. 22 is an exploded perspective view of the distal end cover shown in FIG. 21;

FIG. 22A is a front view of a first cover in the distal end cover shown in FIG. 22;

FIG. 22B is a top view of the first cover in the distal end cover shown in FIG. 22;

FIG. 22C is a bottom view of the first cover in the distal end cover shown in FIG. 22;

FIG. 22D is a side view of the first cover in the distal end cover shown in FIG. 22;

FIG. 22E is another side view of the first cover in the distal end cover shown in FIG. 22;

FIG. 22F is a rear view of the first cover in the distal end cover shown in FIG. 22;

FIG. 22G is a front view of a second cover in the distal end cover shown in FIG. 22;

FIG. 22H is a top view of the second cover in the distal end cover shown in FIG. 22;

FIG. 22I is a bottom view of the second cover in the distal end cover shown in FIG. 22;

FIG. 22J is a side view of the second cover in the distal end cover shown in FIG. 22;

FIG. 22K is another side view of the second cover in the distal end cover shown in FIG. 22;

FIG. 22L is a rear view of the second cover in the distal end cover shown in FIG. 22;

FIG. 23 is a partially enlarged perspective view showing a state in which a distal end cover including a sheath is attached to the distal end portion of the insertion section of the endoscope in the endoscope apparatus in the second embodiment;

FIG. 24 is an exploded perspective view showing a state in which the distal end cover is detached from the distal end portion shown in FIG. 23; and

FIG. 25 is an exploded perspective view of the distal end cover shown in FIG. 24.

### DETAILED DESCRIPTION OF THE PREFERRED EMBODIMENTS

Embodiments of the present invention are explained below with reference to the drawings.

### First Embodiment

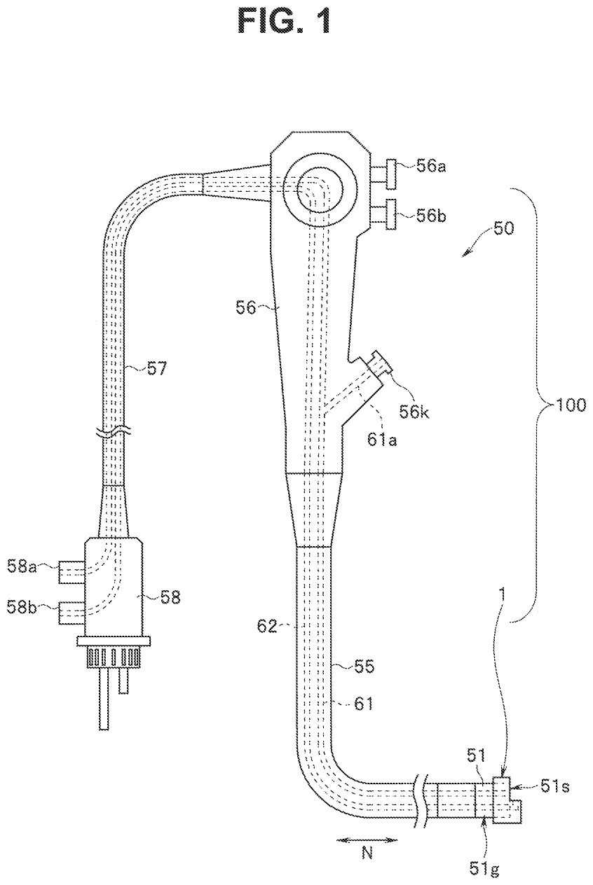

FIG. 1 is a diagram schematically showing a configuration of an endoscope apparatus to which a distal end cover for endoscope in a first embodiment is attached.

As shown in FIG. 1, a main part of an endoscope apparatus 100 includes a distal end cover for endoscope 1 and an endoscope 50.

A main part of the endoscope 50 includes an insertion section 55 formed elongately in a longitudinal axial direction N and to be inserted into a body cavity, an operation section 56 connected consecutively to a proximal end in the longitudinal axial direction N (hereinafter simply referred to as proximal end) of the insertion section 55, a universal cord 57 extended from the operation section 56, and a connector 58 provided at an extension end of the universal cord 57.

The insertion section 55 includes, on a distal end side in the longitudinal axial direction N (hereinafter simply referred to as distal end side), a distal end portion 51 in which an image pickup unit, an illumination unit, and the like not shown in FIG. 1 are provided.

Note that the distal end cover for endoscope (hereinafter simply referred to as distal end cover) 1 is configured to be detachably attachable to the distal end portion 51.

A channel for treatment instrument insertion (hereinafter simply referred to as channel) 61 also functioning as a suction conduit and a fluid passage 62 are provided in the insertion section 55, the operation section 56, the universal cord 57, and the connector 58.

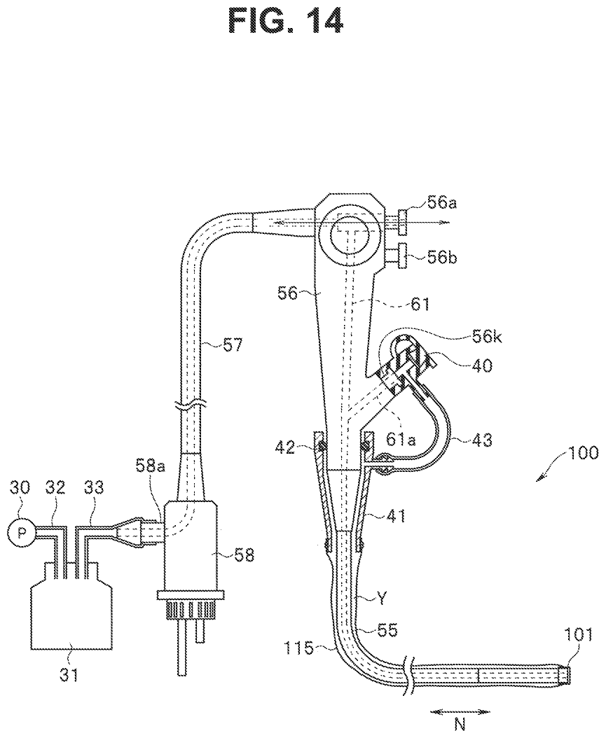

A distal end in the longitudinal axial direction N (hereinafter simply referred to as distal end) of the channel 61 is opened as a distal end opening 61r on a distal end face 51s of the distal end portion 51. A proximal end of the channel 61 communicates with a suction pipe sleeve 58a provided in the connector 58. Note that a suction pump 30 shown in FIG. 14 explained below is configured to be detachably attachable to the suction pipe sleeve 58a.

A conduit 61a divided from a position in the operation section 56 of the channel 61 communicates with a treatment instrument insertion pipe sleeve 56k provided in the operation section 56.

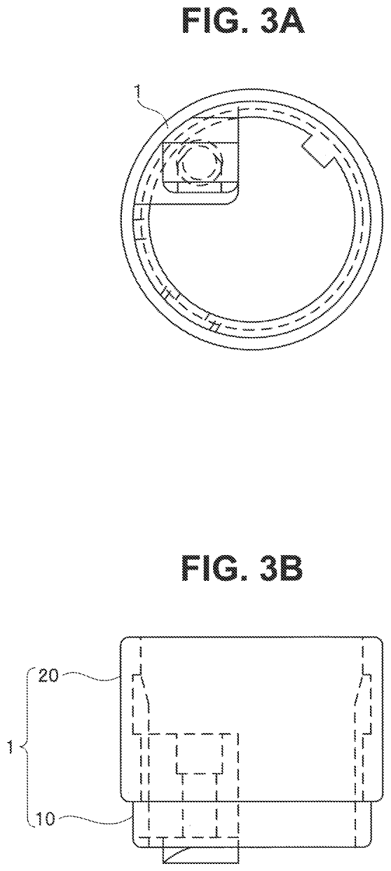

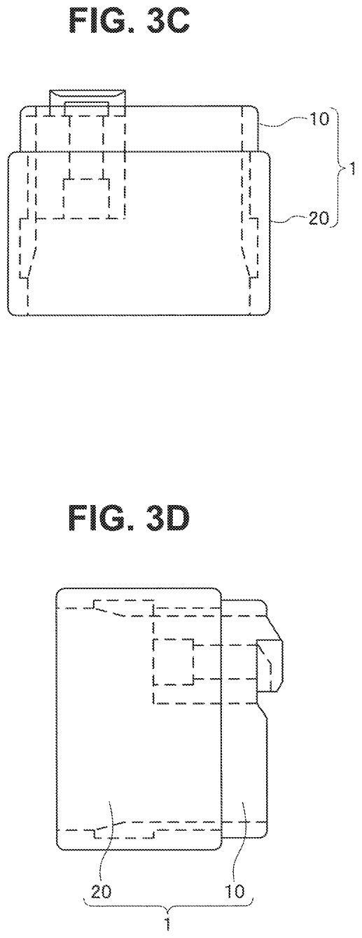

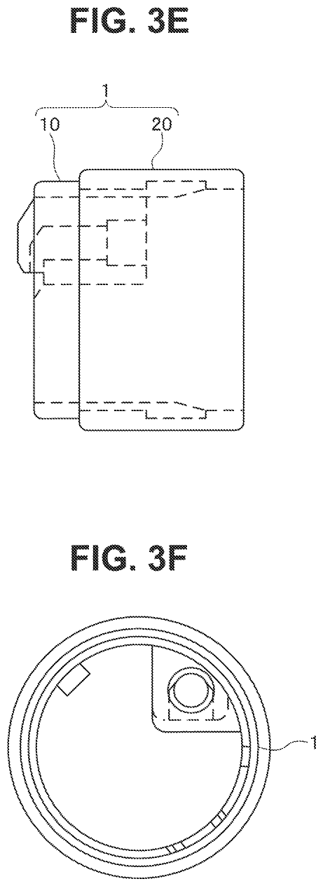

A distal end of the fluid passage 62 is exposed as a distal end opening 62i in a recess 51d formed on the distal end face 51s of the distal end portion 51 as shown in FIG. 3 explained below.

A proximal end of the fluid passage 62 communicates with a supply pipe sleeve 58b provided in the connector 58. Note that a not-shown fluid supply apparatus is configured to be detachably attachable to the supply pipe sleeve 58b.

Further, a suction switching button 56a and a gas feeding and liquid feeding switching button 56b are provided in the operation section 56.

When solid, liquid, and the like in the body cavity are sucked via the channel 61, the suction switching button 56a is operated by an operator to, for example, open and close a communication path to a conduit connected to the suction pump 30.

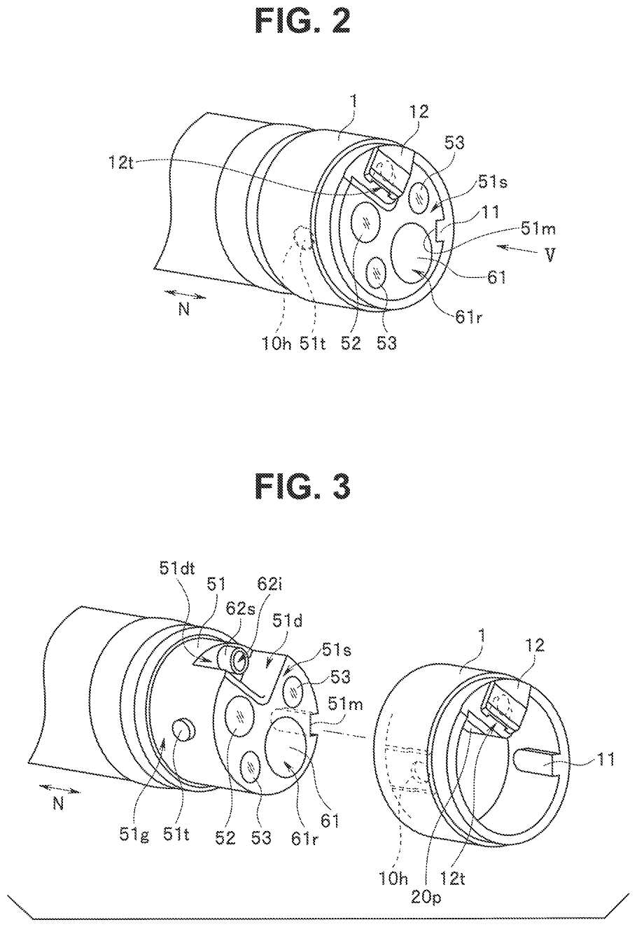

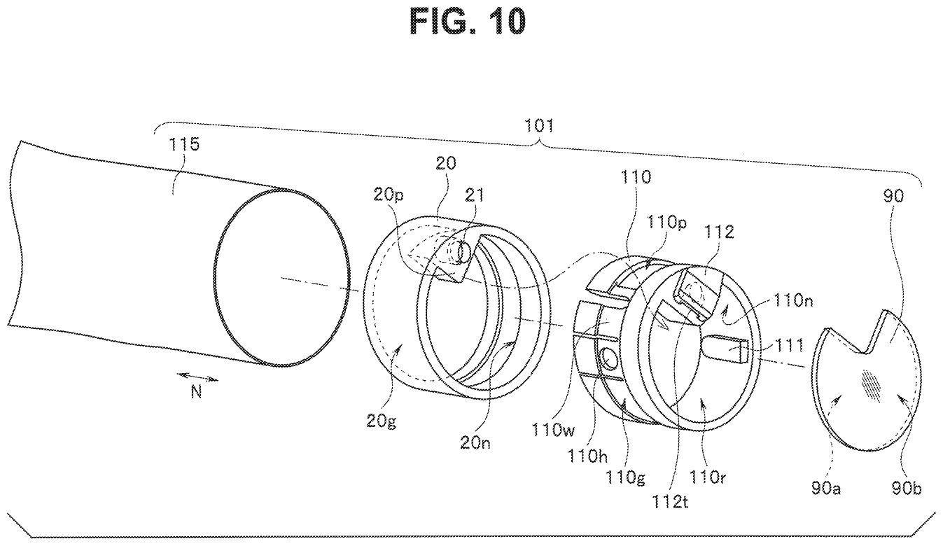

When fluid is supplied to an observation window 52 (see FIG. 2) explained below or a second surface 90b of a light transmitting plate 90 (for both of which, see FIG. 10) including a part located in a visual field range of the observation window 52 via the fluid passage 62, the gas feeding and liquid feeding switching button 56b is operated by the operator to, for example, open and close a communication path to a conduit connected to a not-shown fluid supply apparatus.

Note that, actually, the suction switching button 56a is disposed on a conduit of the channel 61 and the gas feeding and liquid feeding switching button 56b is disposed on a conduit of the fluid passage 62. However, since structures of the switching buttons are the same as structures of well-known switching buttons, detailed explanation of the structures of the switching buttons is omitted.

Subsequently, a configuration of the distal end cover 1 is explained with reference to FIGS. 2 to 15.

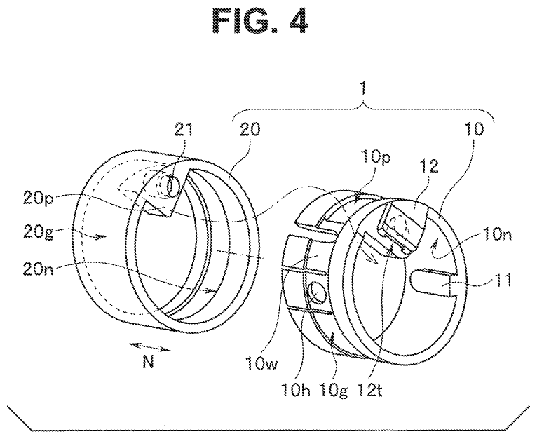

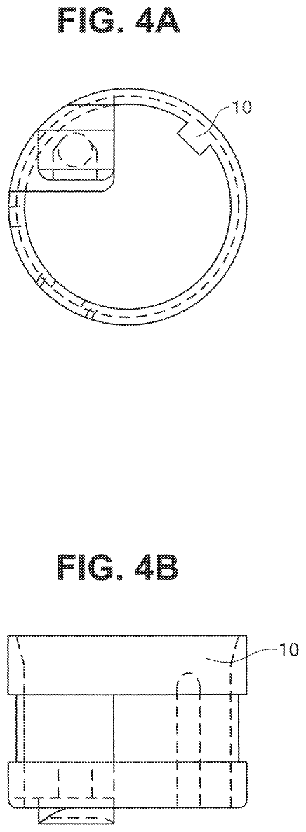

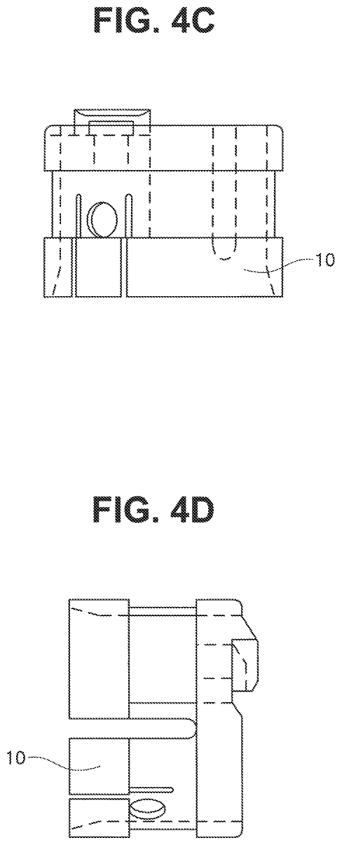

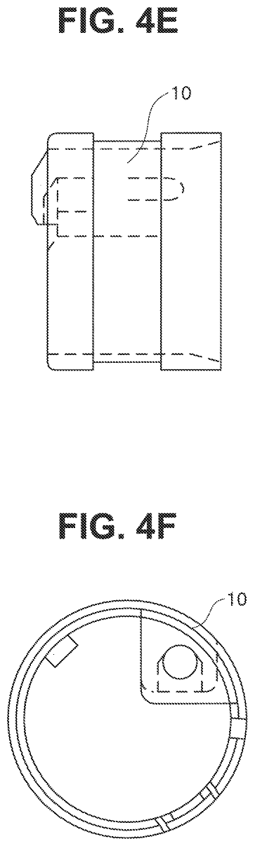

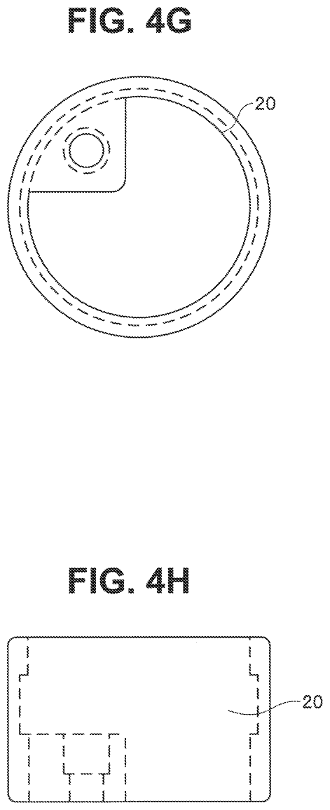

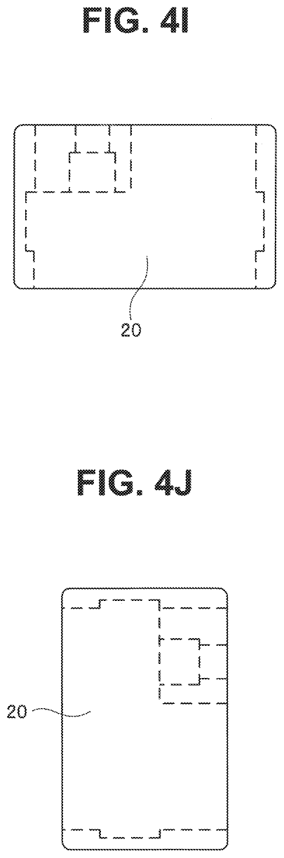

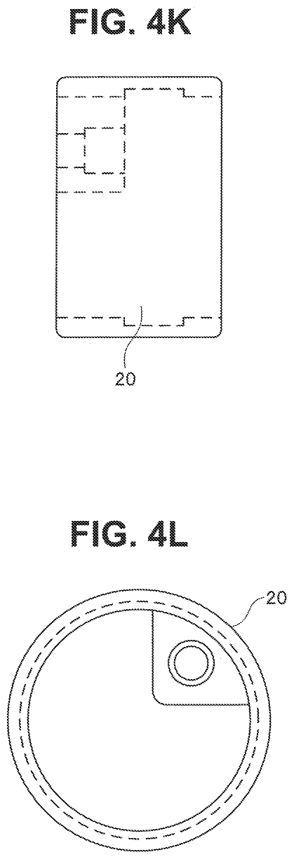

FIG. 2 is a partially enlarged perspective view showing a state in which a distal end cover not including a sheath is attached to the distal end portion of the insertion section of the endoscope shown in FIG. 1. FIG. 3 is an exploded perspective view showing a state in which the distal end cover is detached from the distal end portion shown in FIG. 2, FIGS. 3A to 3F are six views of the distal end cover shown in FIG. 3. FIG. 4 is an exploded perspective view of the distal end cover shown in FIG. 3. FIGS. 4A to 4F are six views of a first cover shown in FIG. 4. FIGS. 4G to 4L are six views of a second cover shown in FIG. 4.

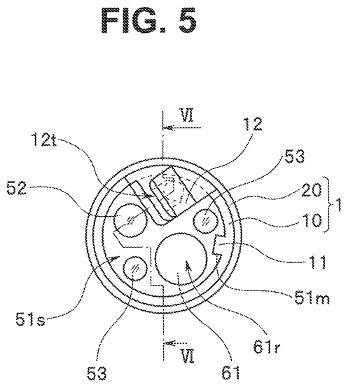

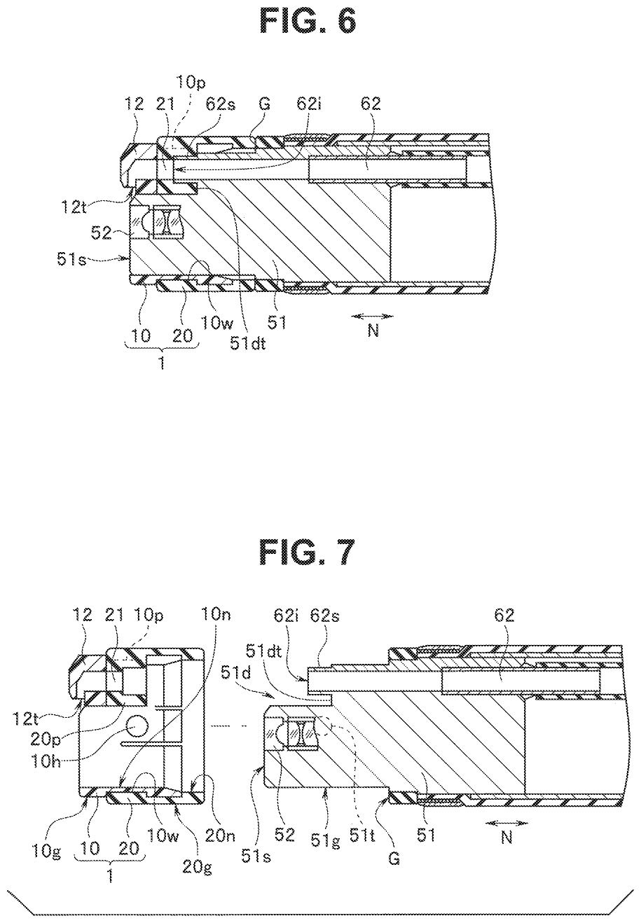

FIG. 5 is a front view of the distal end portion and the distal end cover shown in FIG. 2 viewed in a V direction in FIG. 2. Fig; is a partial cross-sectional view of the distal end portion and the distal end cover taken along a VI-VI line in FIG. 5. FIG. 7 is a partial cross-sectional view showing a state in which the distal end cover is detached from the distal end portion shown in FIG. 6.

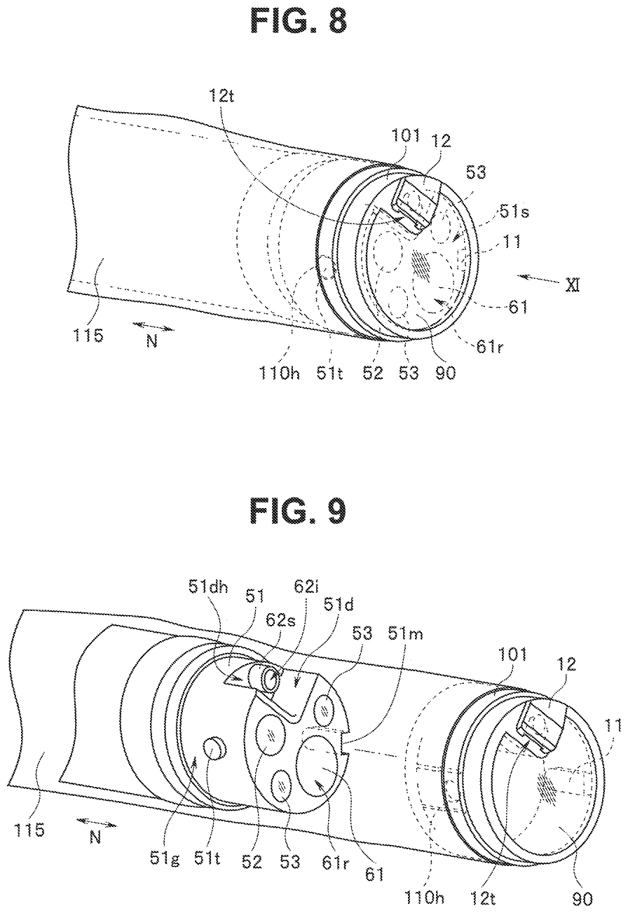

Further, FIG. 8 is a partially enlarged perspective view showing a state in which a distal end cover including a sheath is attached to the distal end portion of the insertion section of the endoscope shown in FIG. 1. FIG. 9 is an exploded perspective view showing a state in which the distal end cover is detached from the distal end portion shown in FIG. 8. FIG. 10 is an exploded perspective view of the distal end cover shown in FIG. 9.

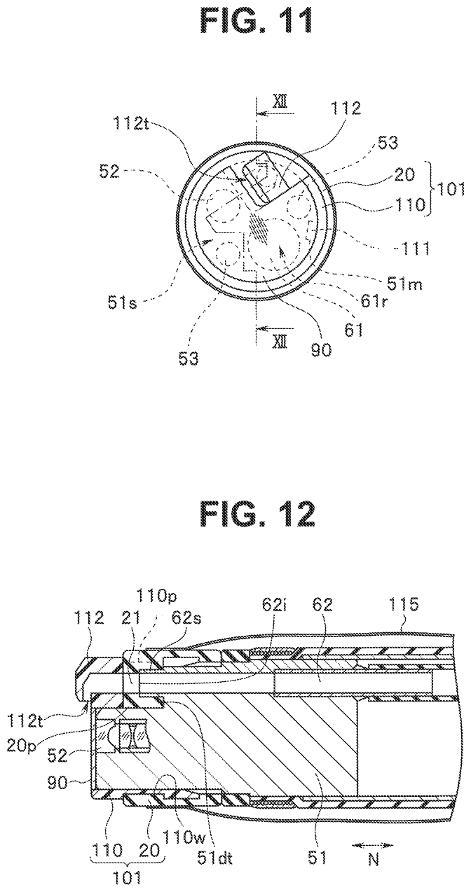

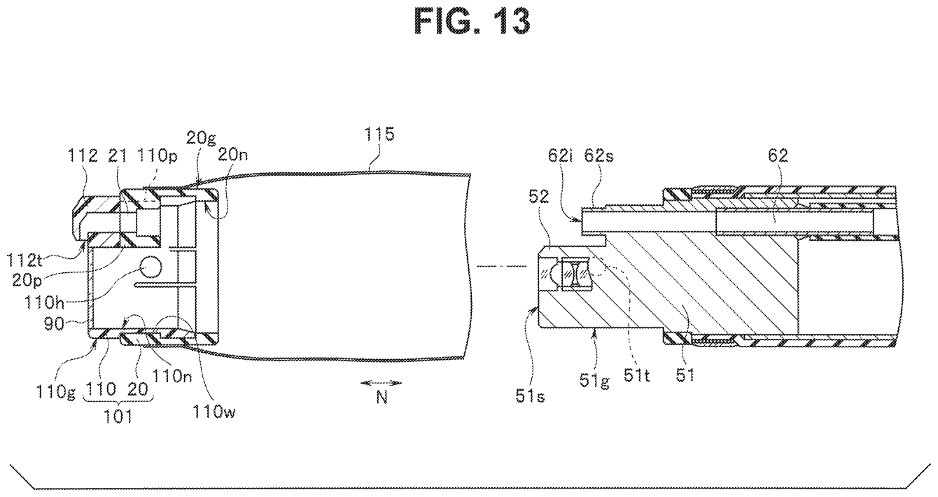

FIG. 11 is a front view of the distal end portion and the distal end cover shown in FIG. 8 viewed in a XI direction in FIG. 8. FIG. 12 is a partial cross-sectional view of the distal end portion and the distal end cover taken along a XII-XII line in FIG. 11. FIG. 13 is a partial cross-sectional view showing a state in which the distal end cover is detached from the distal end portion shown in 12.

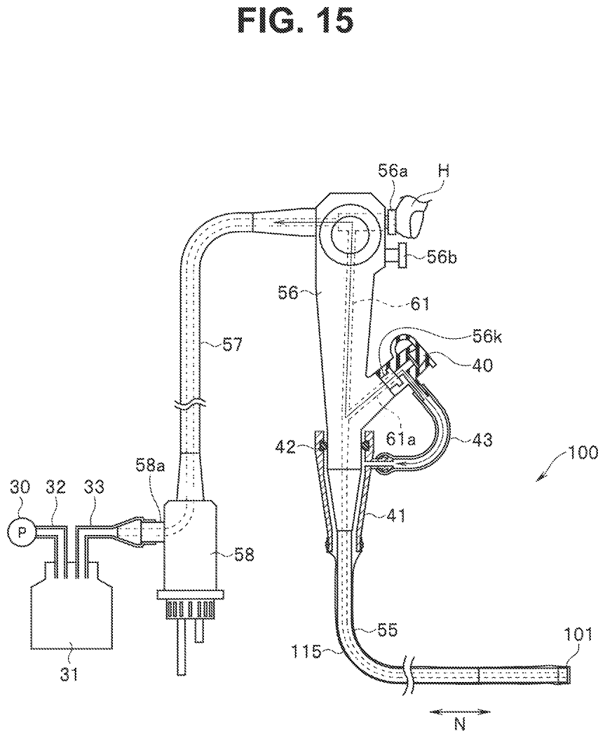

Further, FIG. 14 is a diagram showing a state in which a plug is attached to the treatment instrument insertion pipe sleeve and a suction pump is connected to the suction pipe sleeve in the state in which the distal end cover including the sheath is attached to the distal end portion of the insertion section of the endoscope shown in FIG. 1. FIG. 15 is a diagram showing a state in which the suction switching button shown in FIG. 14 is closed and the sheath adheres through suction via the channel by the suction pump.

As shown in FIGS. 2, 3, and 5 to 7, the distal end cover 1 is configured to be detachably attachable to the distal end portion 51. The distal end cover 1 has a shape shown in FIGS. 3 and 3A to 3F.

In the distal end portion 51, as shown in FIG. 3, the observation window 52, illumination windows 53, and the distal end opening 61r of the channel 61 are provided on the distal end face 51s.

The recess 51d toward a rear in the longitudinal axial direction N (hereinafter simply referred to as rear) is formed on the distal end face 51s. A distal end side 62s of the fluid passage 62 is provided on a bottom surface 51dt of the recess 51d to project to a front in the longitudinal axial direction N (hereinafter simply referred to as front) from the bottom surface 51dt. Consequently, the distal end opening 62i of the fluid passage 62 is exposed in the recess 51d.

Further, a locking pin 5 it is formed on an outer circumferential surface 51g of the distal end portion 51. When the distal end cover 1 is attached to the outer circumferential surface 51g of the distal end portion 51, a locking hole 10h explained below of a first cover 10 is locked to the locking pin 51t as shown in FIG. 2.

As shown in FIG. 3, a locking groove 51m (a first locking section) extending in the longitudinal axial direction N is formed on the outer circumferential surface 51g of the distal end portion 51. When the distal end cover 1 is attached to the outer circumferential surface 51g of the distal end portion 51, a locking protrusion 11 explained below of the first cover 10 engages (fits) in the locking groove 51m as shown in FIGS. 2 and 5.

As shown in FIG. 4, the distal end cover 1 is configured by the first cover 10 and a second cover 20. Note that the first cover 10 has a shape shown in FIGS. 4 and 4A to 4F and the second cover 20 has a shape shown in FIGS. 4G to 4L.

The first cover 10 is formed into a short cylinder in the longitudinal axial direction N from hard resin such as plastic and is configured such that an inner circumferential surface 10n is engageable with the outer circumferential surface 51g of the distal end portion 51.

In the first cover 10, a nozzle 12, a discharge port 12t of which is opposed to the observation window 52 as shown in FIG. 6 when the first cover 10 is attached to the outer circumferential surface 51g of the distal end portion 51 as shown in FIGS. 2, 5, and 6, is provided. The nozzle 12 is a nozzle for spraying fluid to the observation window 52 from the discharge port 12t.

Note that the first cover 10 is formed from the hard resin in order to prevent deformation due to pressure at the time when the fluid is sprayed from the nozzle 1

Further, the locking protrusion 11 (a second locking section) is provided in the longitudinal axial direction N on the inner circumferential surface 10n of the first cover 10.

The locking protrusion 11 is engaged in the locking groove 51m when the first cover 10 is engaged with the outer circumferential surface 51g f the distal end portion 51. Consequently, the first cover 10 is positioned in a turning direction with respect to the distal end portion 51.

A circumferential groove 10w is formed on an outer circumferential surface 10g of the first cover 10. The locking hole 10h passing through the outer circumferential surface 10g and the inner circumferential surface 10n is formed in the groove 10w.

When the first cover 10 is engaged with the outer circumferential surface 51g of the distal end portion 51, the locking pin 51t is locked in the locking hole 10h. Consequently, the first cover 10 is positioned in the turning direction and the longitudinal axial direction N with respect to the distal end portion 51.

Further, a through-hole 10p passing through the outer circumferential surface 10g and the inner circumferential surface 10n is formed in a position where the nozzle 12 in the circumferential direction is provided in the groove 10w of the first cover 10.

The second cover 20 contains a member having more elasticity than the first cover 10, for example, rubber and is formed into a short cylinder in the longitudinal axial direction N.

Further, the second cover 20 is configured such that a part of an inner circumferential surface 20n is engageable to adhere to the outer circumferential surface 10g of the first cover 10 and a part on a proximal end side in the longitudinal axial direction N (hereinafter simply referred to as proximal end side) of the inner circumferential surface 20n is engageable to adhere to an adhesion surface G of the outer circumferential surface 51g.

Note that the second cover 20 is formed from rubber in order to secure watertightness for the distal end portion 51 when the distal end cover 1 is attached to the outer circumferential surface 51g.

A flow channel connecting section 20p projecting in an inner circumferential direction (a radial direction inner side) is provided on the inner circumferential surface 20n of the second cover 20.

In the flow channel connecting section 20p, as shown in FIGS. 4 and 7, a flow channel 21 for causing a distal end and a proximal end of the flow channel connecting section 20p to communicate is provided on the inside.

When the inner circumferential surface 20n of the second cover 20 is engaged with the outer circumferential surface 10g, of the first cover 10 to adhere thereto as shown in FIG. 3, the flow channel connecting section 20p is inserted into the first cover 10 via the through-hole 10p as indicated by an alternate long and short dash line in FIG. 4. As a result, a projecting part on a distal end side of the flow channel 21 is inserted into the nozzle 12, whereby the flow channel 21 communicates with a flow channel in the nozzle 12.

When the distal end cover 1 is attached to the distal end portion 51, as shown in FIG. 6, the distal end side 62s of the fluid passage 62 is inserted into the flow channel 21, whereby the flow channel 21 is caused to communicate with the fluid passage 62.

Consequently, the flow channel 21 circulates, to the nozzle 12, fluid fed from the fluid passage 62. As a result, the fluid is sprayed to the observation window 52 from the discharge port 12t of the nozzle 12.

When the second cover 20 is engaged with the outer circumferential surface 10g of the first cover 10, the flow channel 21 circulates the fluid to the nozzle 12 with an elastic force of the flow channel connecting section 20p of the second cover 20 in a watertight state for the nozzle 12.

Further, when the distal end cover 1 is attached to the outer circumferential surface 51g, the flow channel 21 and the fluid passage 62 are caused to communicate in a watertight state with the elastic force of the flow channel connecting section 20p.

Note that although the second cover 20 is described as engageable with the outer circumferential surface 10g of the first cover 10 in the above explanation, actually, the second cover 20 may be kept engaged after being engaged with the outer circumferential surface 10g of the first cover 10 or both of the first cover 10 and the second cover 20 may be prevented from separating by being bonded.

The first cover 10 and the second cover 20 may be integrally formed as the distal end cover 1.

The insertion section 55 is inserted into the body cavity in a state in which the distal end cover 1 is attached to the distal end portion 51 as explained above.

Thereafter, when the observation window 52 is dirty and observation is difficult, the gas feeding and liquid feeding switching button 56b is operated by the operator, whereby the fluid fed from the fluid passage 62 via the flow channel 21 is sprayed to the observation window 52 from the discharge port 12t of the nozzle 12 and the contaminants are removed. Consequently, a visual field of the observation window 52 is secured.

Note that although the distal end cover 1 is also described as detachably attachable to the outer circumferential surface 51g of the distal end portion 51, actually, since the distal end cover 1 is fixed to the distal end portion 51 by bonding or the like after use, when the distal end cover 1 is detached from the distal end portion 51 after the use, the distal end cover 1 is detached from the distal end portion 51 by being broken.

Thereafter, the distal end cover 1 is discarded and cleaning, disinfection, and sterilization treatment is applied to the endoscope 50.

Further, the second cover 20 is detachably engageable with not only the outer circumferential surface 10g of the first cover 10 shown in FIGS. 2 to 7 but also an outer circumferential surface 110g of a first cover 110 shown in FIGS. 8 to 13. Depending on an embodiment, the second cover 20 may not be detachably engageable with the first cover 110.

A configuration of a distal end cover 101 including the first cover 110 and the second cover 20 is explained below with reference to FIGS. 8 to 15.

Note that, since a configuration of the second cover 20 is the same as the configuration shown in FIGS. 2 to 7, explanation of the configuration is omitted. In this configuration, the endoscope apparatus 100 is configured by the distal end cover 101 and the endoscope 50.

In the configuration shown in FIGS. 8 to 15, the distal end cover 101 is configured by the first cover 110, the second cover 20, the light transmitting plate 90, and a sheath 115.

The first cover 110 is formed into a short cylinder in the longitudinal axial direction N from hard resin such as plastic and has a configuration in which an inner circumferential surface 110n is engageable with the outer circumferential surface 51g of the distal end portion 51.

In the first cover 110, a nozzle 112, a discharge port 1121 of which is opposed to a part located in the visual field range of the observation window 52 in the light transmitting plate 90 as shown in FIG. 11 when the first cover 110 is attached to the outer circumferential surface 51g of the distal end portion 51 as shown in FIGS. 8, 11, and 12, is provided.

The nozzle 112 is a nozzle for spraying fluid from the discharge port 1121 to a part located in the visual field range of the observation window 52 on a second surface 901 explained below of the light transmitting plate 90.

Note that, since the nozzle 112 sprays the fluid to the light transmitting plate 90, a projection amount to the front of the nozzle 112 is larger compared with the nozzle 12 explained above that sprays the fluid to the observation window 52.

Note that the first cover 110 is formed from the hard resin in order to prevent deformation due to pressure at the time when the fluid is sprayed from the nozzle 112.

Further, a locking protrusion 111 is provided in the longitudinal axial direction N on the inner circumferential surface 110n of the first cover 110.

The locking protrusion 111 is engaged in the locking groove 51m when the first cover 110 is attached to the outer circumferential surface 51g of the distal end portion 51. Consequently, the first cover 110 is positioned in a turning direction with respect to the distal end portion 51.

A circumferential groove 110w is formed on the outer circumferential surface 110g of the first cover 110. A locking hole 110h passing through the outer circumferential surface 110g and the inner circumferential surface 110n is formed in the groove 110w.

The locking pin 51t is locked in the locking hole 110h when the first cover 110 is attached to the outer circumferential surface 51g of the distal end portion 51. Consequently, the first cover 110 is positioned in the turning direction and the longitudinal axial direction N with respect to the distal end portion 51.

Further, a through-hole 110p passing through the outer circumferential surface 110g and the inner circumferential surface 110n is formed in a position where the nozzle 112 is provided in a circumferential direction in the groove 110w of the first cover 110.

Note that although the second cover 20 is described as detachably attachable to the outer circumferential surface 110g of the first cover 110 in the above explanation, actually, an engaged state may be continued after the second cover 20 is engaged with the outer circumferential surface 110g of the first cover 110 or both of the first cover 110 and the second cover 20 may be prevented from separating by being bonded.

The first cover 110 and the second cover 20 may be integrally formed as the distal end cover 101.

The sheath 115 is formed from soft and thin resin such as rubber, vinyl, or polyethylene and a distal end of the sheath 115 is water-tightly fixed to an outer circumferential surface 20g of the second cover 20 by, for example, bonding and fixing or sandwiching.

Note that the distal end of the sheath 115 may be water-tightly fixed to the outer circumferential surface 110g of the first cover 110 or may be water-tightly fixed to the respective inner circumferential surfaces 20n and 110n. An inner diameter of the sheath 115 is set to a size for easily covering the insertion section 55.

The sheath 115 covers an outer surface of the insertion section 55 in the longitudinal axial direction N when the distal end cover 101 is attached to the outer circumferential surface 51g of the distal end portion 51 as shown in FIGS. 14 and 15.

The light transmitting plate 90 is formed from a material having light transmissivity and is water-tightly attached to the first cover 110 to cover a front surface opening 110r of the first cover 110 as shown in FIG. 10. Note that the light transmitting plate 90 may be water-tightly attached to the second cover 20.

The light transmitting plate 90 includes a part located in the visual field range of the observation window 52 by closing the front surface opening 110r of the first cover 110 after the attachment.

Further, the light transmitting plane 90 includes a first surface 90a and a second surface 90b. The first surface 90a is a surface opposite to the observation window 52 when the light transmitting plate 90 is attached to the first cover 110. The second surface 90b is a surface located on an opposite side (rear surface side) of the first surface 90a. The second surface 90b is splayed with the fluid from the nozzle 112 and includes a part located in the visual field range of the observation window 52 when the light transmitting plate 90 is attached to the first cover 110.

The flow channel connecting section 20p is inserted into the first cover 110 via the through-hole 110p as indicated by an alternate long and short dash line in FIG. 10 when the inner circumferential surface 20n of the second cover 20 is attached to the outer circumferential surface 110g of the first cover 110 to adhere thereto as shown in FIGS. 9 and 13. As a result, a projecting part on a distal end side of the flow channel 21 is inserted into the nozzle 112, whereby the flow channel 21 communicates with a flow channel in the nozzle 112.

When the distal end cover 101 is attached to the distal end portion 51, as shown in FIG. 12, the distal end side 62s of the fluid passage 62 is inserted into the flow channel 21, whereby the flow channel 21 is caused to communicate with the fluid passage 62.

Consequently, the flow channel 21 circulates, to the nozzle 112, the fluid fed from the fluid passage 62. As a result, the fluid is sprayed to a part located in the visual field range of the observation window 52 of the second surface 90b from the discharge port 112t of the nozzle 112.

When the second cover 20 is engaged with the outer circumferential surface 110g of the first cover 110, the flow channel 21 circulates, with an elastic force of the second cover 20, the fluid to the nozzle 112 in a watertight state for the nozzle 112.

Further, when the distal end cover 101 is attached to the outer circumferential surface 51g, the flow channel 21 and the fluid passage 62 are caused to communicate in a watertight state by an elastic force of the flow channel connecting section 20p.

After the distal end cover 101 is attached to the distal end portion 51 and the insertion section 55 is covered by the sheath 115 as shown in FIG. 14, to cause the sheath 115 to adhere to the outer surface of the insertion section 55, first, a sheath pipe sleeve 41 is attached to a proximal end of the sheath 115 and is water-tightly and air-tightly connected to an outer circumferential surface of the operation section 56 via an O-shaped ring 42.

As a result, a proximal end of a space Y in the sheath 115 is closed by the O-shaped ring 42 and a distal end of the space Y is closed by the distal end cover 101.

Thereafter, a rubber plug 40 is attached to the treatment instrument insertion pipe sleeve 56k and a space communicating with the conduit 61a in the rubber plug 40 and a space in the sheath pipe sleeve 41 are caused to communicate using a branch tube 43.

Subsequently, the suction pump 30 is connected to the suction pipe sleeve 58a via tubes 32 and 33 and a tank 31.

Note that, in a state shown in FIG. 14, even if the suction pump 30 is driven, since the suction switching button 56a is released, the suction pump 30 is only sucking gas outside the endoscope apparatus 100 via the channel 61 and the suction switching button 56a.

At this time, since the distal end opening 61r of the channel 61 is closed by the distal end cover 101, an airtight state is retained.

Thereafter, when an opening of the suction switching button 56a is closed by a finger H of the operator as shown in FIG. 15, the suction pump 30 sucks gas in the space Y in the sheath 115 via spaces in the channel 61, the conduit 61a, the branch tube 43, and the sheath pipe sleeve 41. As a result, the sheath 115 adheres to the outer surface of the insertion section 55.

Note that, as explained above, when the sheath 115 is caused to adhere to the outer surface of the insertion section 55, since adhesion work can be performed by the suction pump 30 used for normal suction, it is unnecessary to separately prepare a special suction apparatus or jig.

Further, with a simple configuration using the suction pump 30 and the channel 61, it is possible to cause the sheath 115 to adhere to the outer surface of the insertion section 55 at low cost.

The insertion section 55 is inserted into the body cavity in a state in which the sheath 115 is caused to adhere to the outer surface of the insertion section 55.

Thereafter, when a part located in the visual field range of the observation window 52 is dirty and observation is difficult on the second surface 90b of the light transmitting plate 90, the gas feeding and liquid feeding switching button 56b is operated by the operator, whereby fluid is supplied from the discharge port 112t of the nozzle 112 to the second surface 90b, the contaminants are removed, and a visual field of the observation window 52 is secured.

Note that although the distal end cover 101 is described as detachably attachable to the outer circumferential surface 51g of the distal end portion 51, actually, when the distal end cover 101 is detached from the distal end portion 51 after use, the distal end cover 101 is detached from the distal end portion 51 by being broken. Thereafter, the distal end cover 101 is discarded and cleaning, disinfection, and sterilization treatment is applied to the entire endoscope 50.

As explained above, in the present embodiment, the distal end cover 1 not including the sheath 115 and the distal end cover 101 including the sheath 115 are described as selectively detachably attachable to the distal end portion 51 of the insertion section 55 of one endoscope 50.

The distal end cover 1 is described as, when being attached to the distal end portion 51 by the flow channel 21 of the second cover 20, being capable of causing the fluid passage 62 and the nozzle 12 to water-tightly communicate with the elastic force of the flow channel connecting section 20p and spraying the fluid to the observation window 52.

Further, the distal end cover 101 is described as, when being attached to the distal end portion 51 by the flow channel 21 of the second cover 20, being capable of causing the fluid passage 62 and the nozzle 112 to water-tightly communicate with the elastic force of the flow channel connecting section 20p and spraying the fluid to the part located in the visual field range of the observation window 52 on the second surface 90b of the light transmitting plate 90.

With such a configuration, if the distal end cover 1 is attached to the distal end portion 51, the endoscope 50 can be used in the same manner as a conventional endoscope in which the insertion section 55 is not covered by a sheath. Also, if the distal end cover 101 is attached to the distal end portion 51, the endoscope 50 can be used as a conventional endoscope in which the insertion section 55 is covered by a sheath.

In other words, only by changing the distal end covers in one endoscope 50, it is possible to properly use, according to clinical case content or the like, a method of use for covering the insertion section 55 with the sheath 115 and a method of use for not covering the insertion section 55 with the sheath 115.

Accordingly, a user does not need to prepare two endoscopes, that is, an endoscope in which the insertion section 55 needs to be covered by the sheath 115 and an endoscope in which the insertion section 55 does not need to be covered by the sheath 115.

In both of the distal end cover 1 and the distal end cover 101, the flow channel 21 for causing the fluid passage 62 and the nozzle 12 to communicate and the flow channel 21 for causing the fluid passage 62 and the nozzle 112 to communicate are provided in the second cover 20 in the distal end cover 1 and the distal end cover 101.

For this reason, since the flow channel 21 provided in the distal end cover is shorter compared with a distal end cover in which a flow channel is provided in parallel to an insertion section in the longitudinal axial direction N after being attached to a conventional distal end portion, the flow channel 21 is less easily buckled and can supply, to the nozzles 12 and 112, the fluid from the fluid passage 62 without a gas feeding and liquid feeding failure.

Further, since the nozzles 12 and 112 can be brought closer to a visual field center of the observation window 52 than in a distal end cover in which a fluid passage is provided on an outer side in a radial direction of an insertion section after a conventional distal end cover is attached, fluid supply power to the observation window 52 and a part located in the visual field range of the observation window 52 in the light transmitting plate 90 can be increased and a draining property is improved.

From the above, an object is to provide the distal end covers for endoscope 1 and 101 and the endoscope apparatus 100 in which the fluid supply nozzles 12 and 112 for removing, with the fluid supplied from the fluid passage 62, a target object disturbing the visual field of the observation window 52 of the endoscope 50 are provided for the insertion section 55 of the one endoscope 50 usable in both cases of a form of use in which the insertion section 55 is covered by the sheath 115 and a form of use in which the insertion section 55 is not covered by the sheath 115 and including the fluid passage 62.

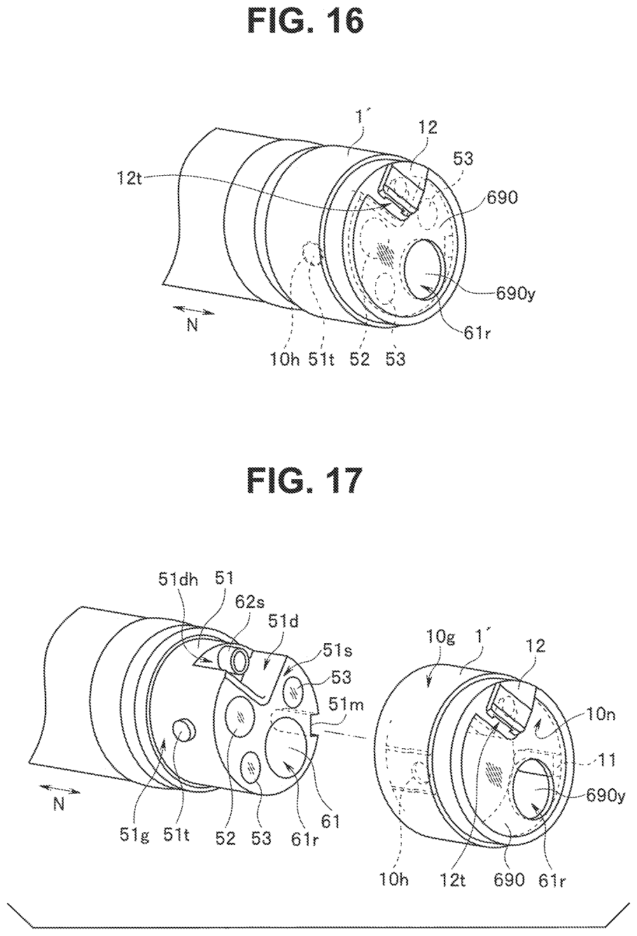

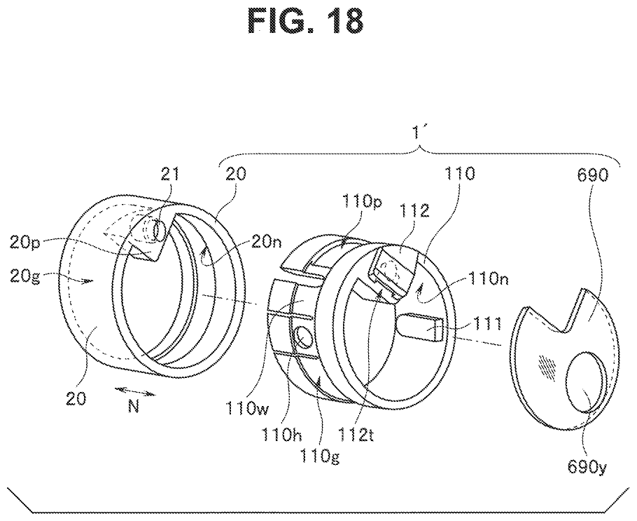

Note that a modification is explained below with reference to FIGS. 16 to 18. FIG. 16 is a partially enlarged perspective view showing a state in which a distal end cover not including a sheath and including a light transmitting plate is attached to the distal end portion of the insertion section of the endoscope shown in FIG. 1. FIG. 17 is an exploded perspective view showing a state in which the distal end cover is detached from the distal end portion shown in FIG. 16. FIG. 18 is an exploded perspective view of the distal end cover shown in FIG. 17.

In the present embodiment explained above, the distal end cover 1 is described as being configured from the first cover 10 and the second cover 20. The light transmitting plate 90 is described as being provided in the distal end cover 101.

Alternatively, as shown in FIGS. 16 to 18, a light transmitting plate 690 may be provided in a distal end cover 1′ in which the sheath 115 is not used. In this case, the endoscope apparatus 100 is configured from the distal end cover and the endoscope 50.

In other words, the distal end cover in which the sheath 115 is not used may be configured from the first cover 110 and the second cover 20 used in the distal end cover 101 and the light transmitting plate 690.

Note that, at this time, if a distal end side of the first cover 110 is closed by the light transmitting plate 690, the distal end opening 61r of the channel 61 is also closed, although the sheath 115 is not used. Therefore, an opening 690y only has to be formed in a position opposed to the distal end opening 61r of the channel 61 in the light transmitting plate 690.

With such a configuration, although, in the present embodiment explained above, the first covers are different in the distal end cover 1 in which the sheath 115 is not used and the distal end cover 101 in which the sheath 115 is used, in the distal end cover 1′, according to whether the light transmitting plate 690 is used or the light transmitting plate 90 is used, the first cover 110 can be used in common when the sheath 115 is not used and when the sheath 115 is used. Note that the other effects are the same as the effects of the present embodiment explained above.

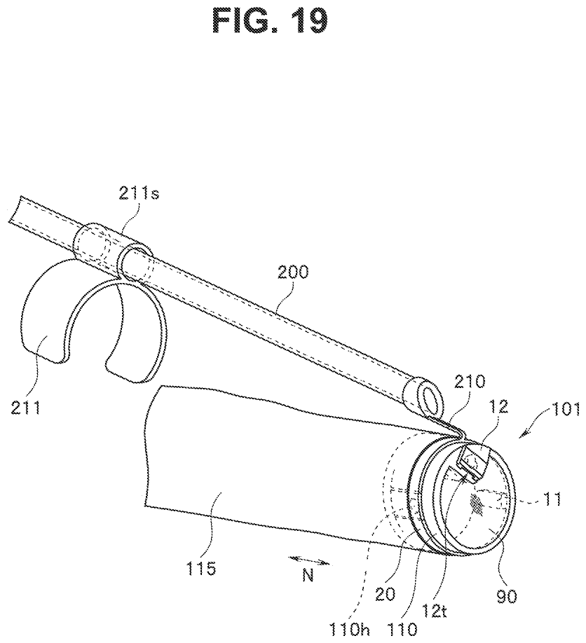

Another modification is explained below with reference to FIG. 19. FIG. 19 is a perspective view showing a modification in which a channel is externally attached to the first cover in the distal end cover shown in FIG. 8.

As shown in FIG. 8 referred to above, in the state in which the distal end cover 101 is attached to the distal end portion 51, the distal end opening 61r of the channel 61 is closed by the light transmitting plate 90 and, as shown in FIG. 15, the plug 40 is attached to the treatment instrument insertion pipe sleeve 56k. Therefore, a treatment instrument cannot be inserted into and removed from the body cavity via the channel 61 and liquid, solid, and the like in the body cavity cannot be sucked via the channel 61.

Accordingly, as shown in FIG. 19, an external channel 200 extended in the longitudinal axial direction N outside the insertion section 55 and different from the flow channel 21 may be fixed to, for example, the first cover 110 via a coupling member 210 having flexibility.

Note that the coupling member 210 is fixed to the first cover 110 by welding or the like. The coupling member 210 may be fixed to the second cover 20.

The external channel 200 is located near the sheath 115 and in the longitudinal axial direction N because halfway positions in the longitudinal axial direction N are inserted in the longitudinal axial direction N respectively through slide sections 211s of a plurality of locking sections 211 locked to the outer surface of the insertion section 55 via the sheath 115.

Note that a proximal end of the external channel 200 is connected to another suction pump different from the suction pump 30 explained above.

With such a configuration, even if the distal end opening 61r of the channel 61 is closed by the light transmitting plate 90, a treatment instrument can be inserted into and removed from the body cavity and liquid, solid, and the like in the body cavity can be sucked via the external channel 200. Note that the other effects are the same as the effects of the present embodiment explained above.

### Second Embodiment

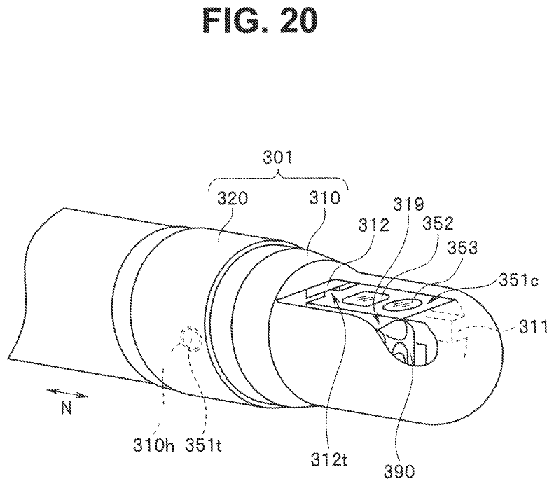

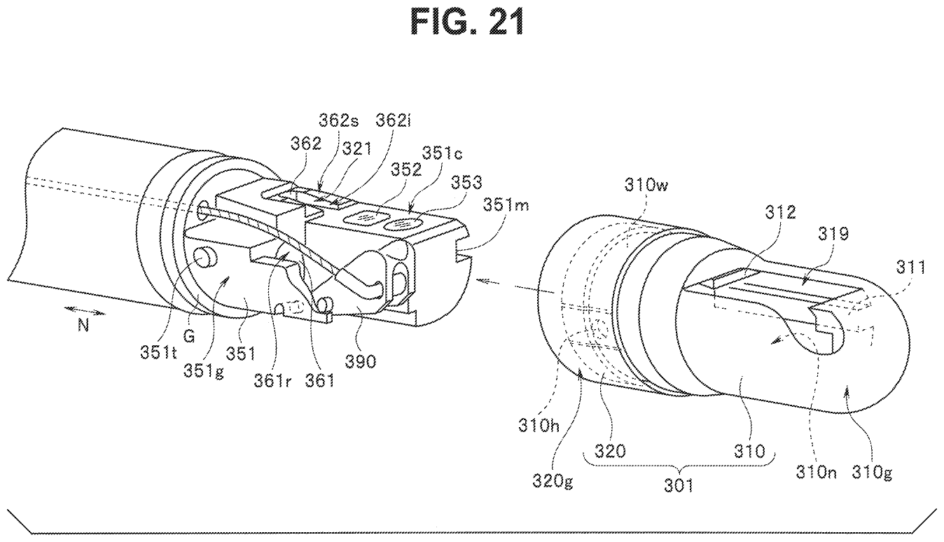

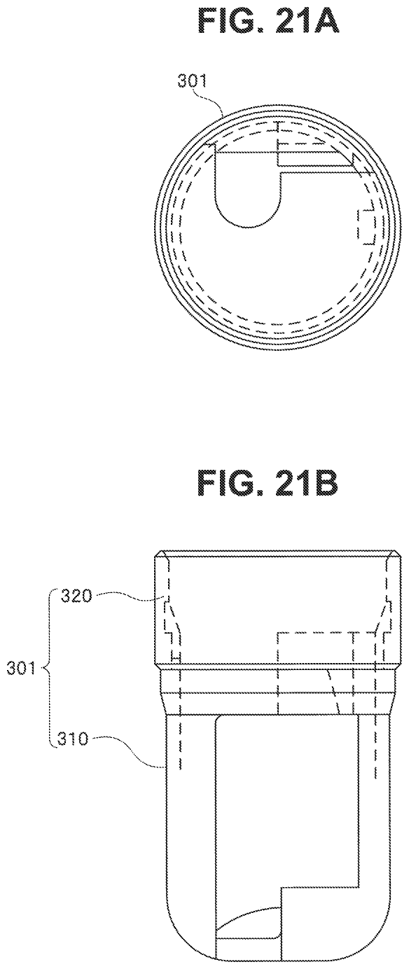

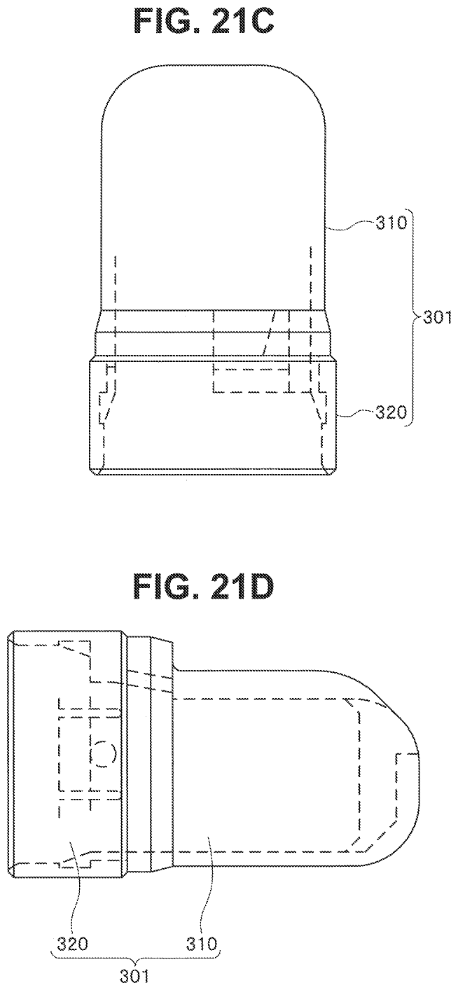

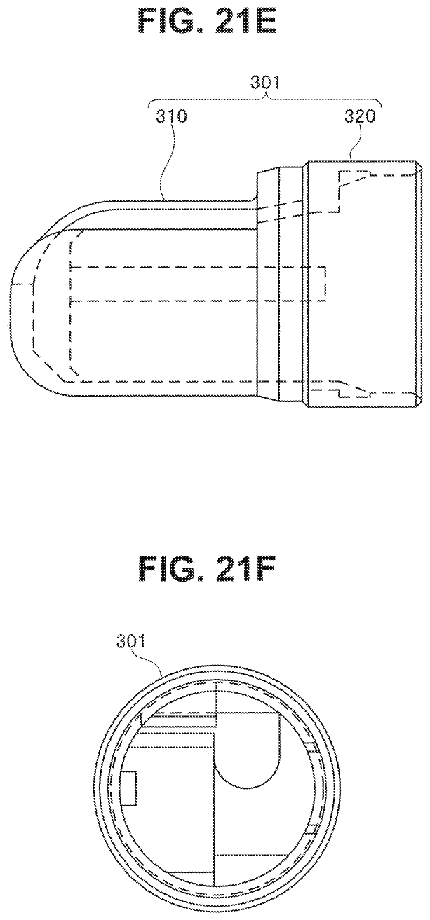

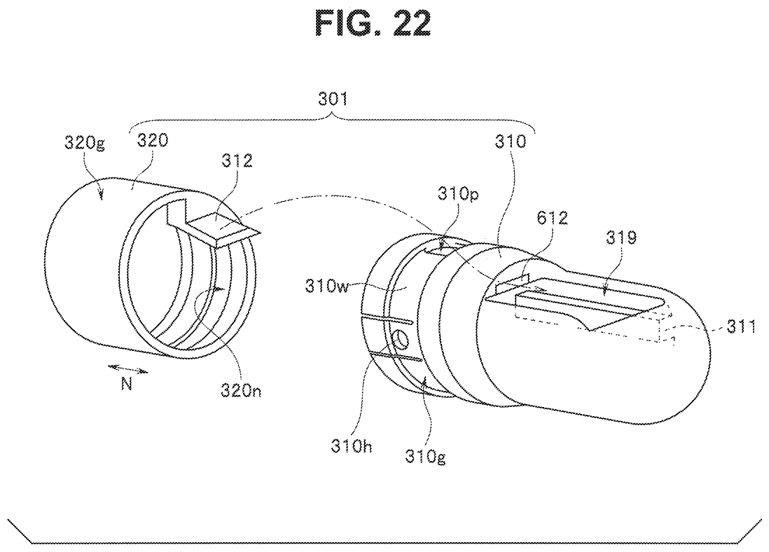

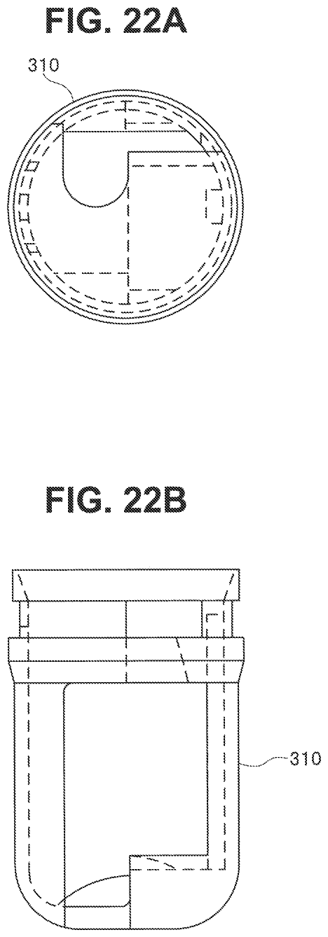

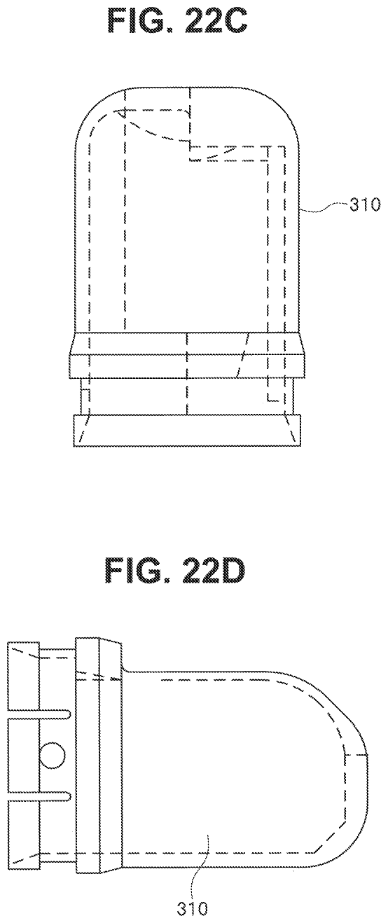

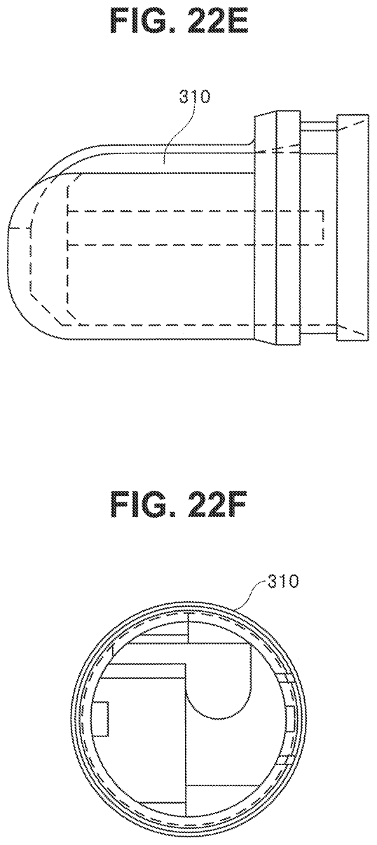

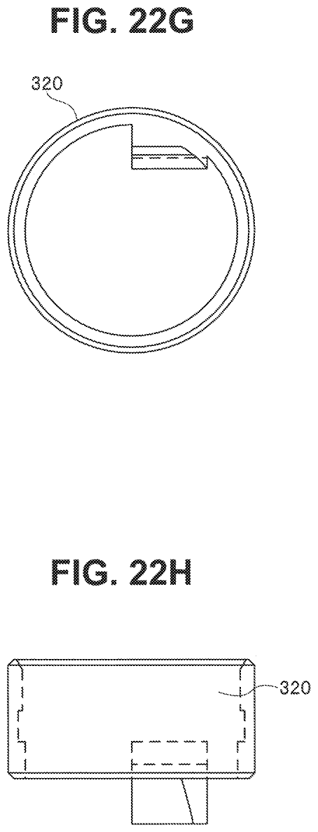

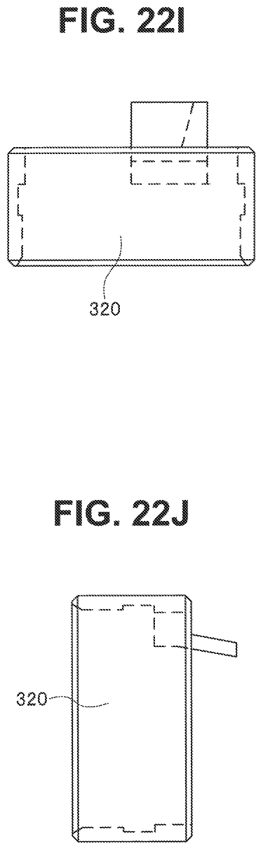

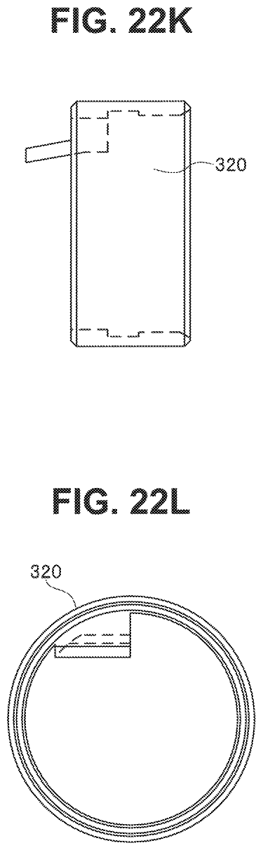

FIG. 20 is a partially enlarged perspective view showing a state in which a distal end cover not including a sheath is attached to a distal end portion of an insertion section of an endoscope in an endoscope apparatus in a second embodiment. FIG. 21 is an exploded perspective view showing a state in which the distal end cover is detached from the distal end portion shown in 20, FIGS. 21A to 21F are six views of the distal end cover shown in FIG. 21. FIG. 22 is an exploded perspective view of the distal end cover shown in FIG. 21. FIGS. 22A to 22F are six views of a first cover shown in FIG. 22. FIGS. 22G to 22L are six views of a second cover shown in FIG. 22.

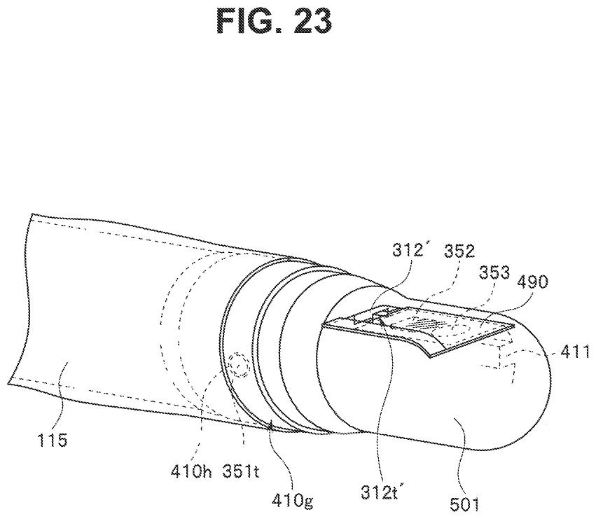

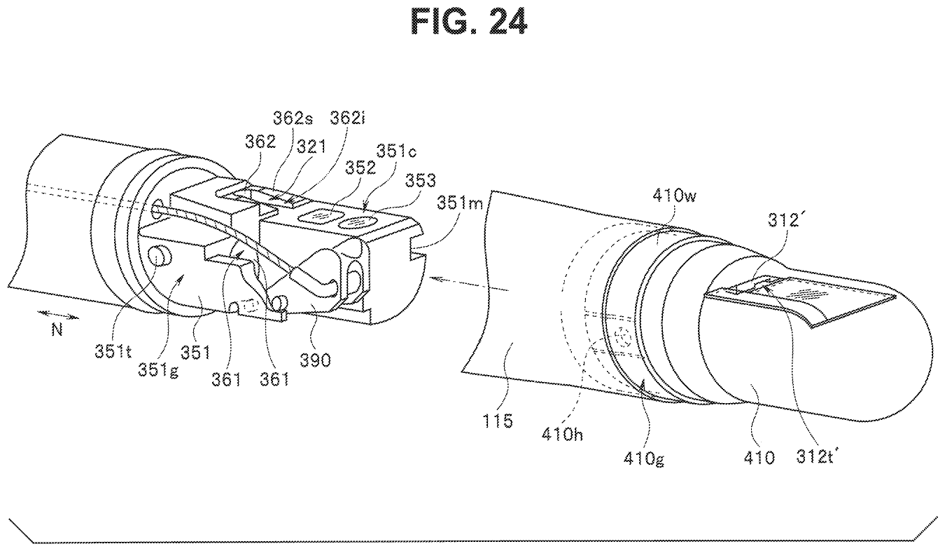

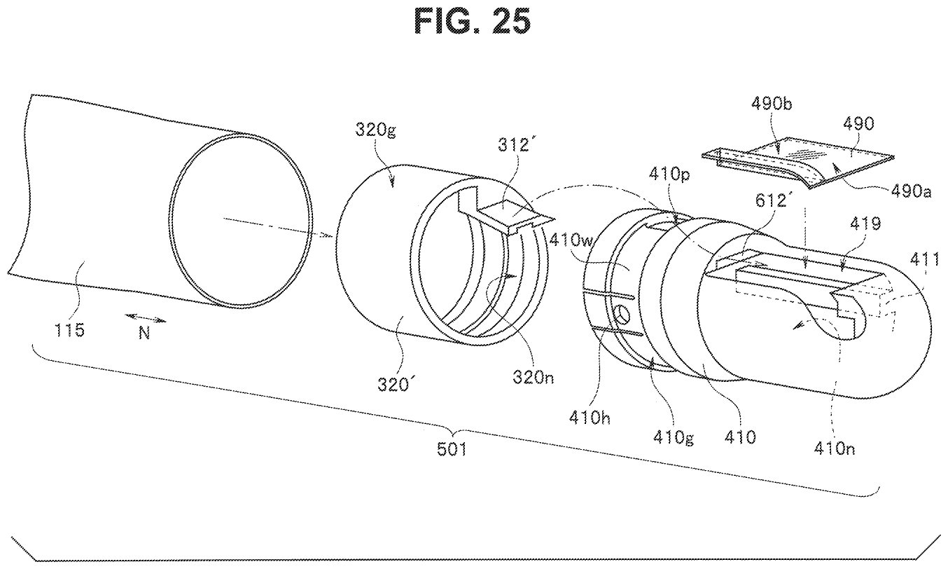

FIG. 23 is a partially enlarged perspective view showing a state in which a distal end cover including a sheath is attached to the distal end portion of the insertion section of the endoscope in the endoscope apparatus in the present embodiment. FIG. 24 is an exploded perspective view showing a state in which the distal end cover is detached from the distal end portion shown in FIG. 23. FIG. 25 is an exploded perspective view of the distal end cover shown in FIG. 24.

In configurations of the endoscope apparatus and the distal end cover in the second embodiment, compared with the configurations of the endoscope apparatus and the distal end cover in the first embodiment shown in FIGS. 1 to 15 explained above, the endoscope is configured from a side view-type endoscope and a shape of the distal end cover is different accordingly.

Accordingly, only this difference is explained. The same components as the components in the first embodiment are denoted by the same reference numerals and signs and explanation of the components is omitted.

As shown in FIGS. 20 to 22, a distal end cover 301 of the endoscope apparatus 100 is detachably attachable to a distal end portion 351 of the insertion section 55. The distal end cover 301 has a shape shown in FIGS. 21 and 21A to 21F.

As shown in FIG. 21, the distal end portion 351 is formed in a shape obtained by cutting out a part of an outer circumferential surface 351g. An observation window 352 and an illumination window 353 are provided in a cutout surface 351c.

In other words, in the present embodiment, the endoscope 50 is configured from a so-called side view-type endoscope that observes a direction crossing the longitudinal axial direction N.

A known treatment instrument raising base (forceps elevator) 390 is provided in the distal end portion 351. A distal end opening 361r of a channel 361 is exposed at the distal end portion 351 to be opposed to the treatment instrument raising base 390 in the longitudinal axial direction N. Note that the channel 361 has the same function as the function of the channel 61 in the first embodiment explained above.

Further, at the distal end portion 351, a distal end opening 362i of a fluid passage 362 is exposed to be opposed to the observation window 352 in the longitudinal axial direction N. Note that the fluid passage 362 has the same function as the function of the fluid passage 62 in the first embodiment explained above.

A locking pin 351t is formed on the outer circumferential surface 351g of the distal end portion 351. When the distal end cover 301 is attached to the outer circumferential surface 351g of the distal end portion 351, a locking hole 310h explained below of a first cover 310 is locked to the locking pin 351t as shown in FIG. 20.

A locking groove 351m extending in the longitudinal axial direction N is formed on the outer circumferential surface 351g of the distal end portion 351. When the distal end cover 301 is attached to the outer circumferential surface 351g of the distal end portion 351, a locking protrusion 311 explained below of the first cover 310 engages in the locking groove 351m as shown in FIG. 20.

As shown in FIG. 22, the distal end cover 301 is configured by the first cover 310 and a second cover 320. Note that the first cover 310 has a shape shown in FIGS. 22 and 22A to 22F and the second cover 320 has a shape shown in FIGS. 22G to 22L.

The first cover 310 is formed from hard resin such as plastic in a cap shape that is elongated in the longitudinal axial direction N and closed at a distal end. The first cover 310 has a configuration in which an inner circumferential surface 310n is engageable with the outer circumferential surface 351g of the distal end portion 351.

In a part of outer circumferential surface 310g of the first cover 310, an opening 319 is formed as shown in FIGS. 20 and 21. The opening 319 causes the observation window 352, the illumination window 353, and the treatment instrument raising base 390 to be exposed to an outside when the first cover 310 is engaged with the distal end portion 351.

Further, the locking protrusion 311 is provided in the longitudinal axial direction N on the inner circumferential surface 310n of the first cover 310.

The locking protrusion 311 is engaged in the locking groove 351m when the first cover 310 is engaged with the outer circumferential surface 351g of the distal end portion 351. Consequently, the first cover 310 is positioned in a turning direction with respect to the distal end portion 351.

A circumferential groove 310w is formed on the outer circumferential surface 310g of the first cover 310. The locking hole 310h passing through the outer circumferential surface 310g and the inner circumferential surface 310n is formed in the groove 310w.

The locking pin 351t is locked in the locking hole 310h when the first cover 310 is engaged with the outer circumferential surface 351g of the distal end portion 351. Consequently, the first cover 310 is positioned in the turning direction and the longitudinal axial direction N with respect to the distal end portion 351.

Further, a through-hole 310p passing through the outer circumferential surface 310g and the inner circumferential surface 310n is formed in a position where the opening 319 is provided in a circumferential direction in the groove 310w of the first cover 310.

The second cover 320 is formed from a member having more elasticity than the first cover 310, for example, rubber and formed into a short cylinder in the longitudinal axial direction N and is engageable such that a part of an inner circumferential surface 320n adheres to the outer circumferential surface 310g of the first cover 310 and is engageable such that a proximal end side part of the inner circumferential surface 320n adheres to a surface G (see FIG. 21) of the outer circumferential surface 351g.

Note that the second cover 320 is formed from the rubber in order to secure watertightness for the distal end portion 351 when the distal end cover 1 is attached to the outer circumferential surface 351g.

A flow channel connecting section 312 projecting in an inner circumferential direction (a radial direction inner side) and forward is provided on the inner circumferential surface 320n of the second cover 320.

When the inner circumferential surface 320n of the second cover 320 is engaged with the outer circumferential surface 310g of the first cover 310 to adhere thereto as shown in FIG. 21, the flow channel connecting section 312 is inserted into the first cover 310 via the through-hole 310p as indicated by an alternate long and short dash line in FIG. 22. As a result, the flow channel connecting section 312 is located at a proximal end of the opening 319.

Note that when the second cover 320 is engaged with the outer circumferential surface 310g of the first cover 310, the flow channel connecting section 312 adheres to, with an elastic force, the inner circumferential surface 310n of the first cover 310 and both surfaces of a distal end side inclined section 362s (see FIG. 21) in a watertight state.

In this state, when the distal end cover 301 is attached to the outer circumferential surface 351g of the distal end portion 51 as shown in FIG. 20, the flow channel connecting section 312 closes an upper part of the distal end side inclined section 362s of the fluid passage 362 exposed at the distal end portion 351. Therefore, the distal end opening 362i facing the opening 319 at a proximal end of the opening 319 is formed as a discharge port 312t of the fluid passage 362 by the flow channel connecting section 312. The fluid passage 362, the upper part of which is closed by the flow channel connecting section 312, configures a flow channel 321.

In other words, the flow channel connecting section 312 configures a nozzle opposed to the observation window 352. Note that a shape of the flow channel connecting section 312 is not limited to a nozzle shape shown in FIG. 22. In the following explanation, the nozzle is also denoted by reference numeral 312.

The nozzle 312 is a nozzle for spraying fluid to the observation window 352 from the discharge port 312t.

Note that the first cover 310 is formed from the hard resin as explained above in order to prevent deformation due to pressure at the time when the fluid is sprayed from the nozzle 312.

Note that although the second cover 320 is described as engageable with the outer circumferential surface 310g of the first cover 310 in the above explanation, actually, the second cover 320 may be kept engaged after being engaged with the outer circumferential surface 310g of the first cover 310 or both of the first cover 310 and the second cover 320 may be prevented from separating by being bonded.

The first cover 310 and the second cover 320 may be integrally formed as the distal end cover 301.

The insertion section 55 is inserted into the body cavity in a state in which the distal end cover 301 is attached to the distal end portion 351 as explained above.

Thereafter, when the observation window 352 is dirty and observation is difficult, the gas feeding and liquid feeding switching button 56b is operated by the operator, whereby the fluid fed from the fluid passage 362 and the flow channel 321 is sprayed to the observation window 352 from the discharge port 312t of the nozzle 312 and the contaminants are removed. Consequently, a visual field of the observation window 352 is secured.

Note that although the distal end cover 301 is also described as detachably attachable to the outer circumferential surface 351g of the distal end portion 351, actually, when the distal end cover 301 is detached from the distal end portion 351 after use, the distal end cover 301 is detached from the distal end portion 351 by being broken.

Thereafter, the distal end cover 301 is discarded and cleaning, disinfection, and sterilization treatment is applied to the entire endoscope 50.

Further, the second cover 320 is detachably engageable with not only the outer circumferential surface 310g of the first cover 310 shown in FIGS. 20 to 22 but also an outer circumferential surface 410g of a first cover 410 shown in FIGS. 23 to 25. Depending on an embodiment, the second cover 320 may not be detachably engageable with the first cover 310.

A configuration of a distal end cover 501 including the first cover 410 and a second cover 320′ is explained below with reference to FIGS. 23 to 25.

Note that, since a configuration of the second cover 320′ is the same as the configuration shown in FIGS. 20 to 22, explanation of the configuration is omitted. In this configuration, the endoscope apparatus 100 is configured by the distal end cover 501 and the endoscope 50.

In the configuration shown in FIGS. 23 to 25, the distal end cover 501 is configured by the first cover 410, the second cover 320′, a light transmitting plate 490, and the sheath 115.

The first cover 410 is formed in a cap shape extending in the longitudinal axial direction N from hard resin such as plastic and has a configuration in which an inner circumferential surface 410n is engageable with the outer circumferential surface 351g of the distal end portion 351.

An opening 419 for exposing the observation window 352, the illumination window 353, and the treatment instrument raising base 390 as shown in FIG. 23 when the distal end portion 351 is engaged with a part of the outer circumferential surface 410g of the first cover 410 is formed.

Further, a locking protrusion 411 is provided in the longitudinal axial direction N on the inner circumferential surface 410n of the first cover 410.

The locking protrusion 411 is engaged in the locking groove 351m when the first cover 410 is engaged with the outer circumferential surface 351g of the distal end portion 351. Consequently, the first cover 410 is positioned in a turning direction with respect to the distal end portion 351.

A circumferential groove 410w is formed on the outer circumferential surface 410g of the first cover 410. A locking hole 410h passing through the outer circumferential surface 410g and the inner circumferential surface 410n is formed in the groove 410w.

The locking pin 351t is locked in the locking hole 410h when the first cover 410 is engaged with the outer circumferential surface 351g of the distal end portion 351. Consequently, the first cover 410 is positioned in the turning direction and the longitudinal axial direction N with respect to the distal end portion 351.

Further, a through-hole 410p passing through the outer circumferential surface 410g and the inner circumferential surface 410n is formed in a position where the opening 419 in the circumferential direction is provided in the groove 410w of the first cover 410.

Note that although the second cover 320′ is described as engageable with the outer circumferential surface 410g of the first cover 410 in the above explanation, actually, the second cover 320′ may be kept engaged after being engaged with the outer circumferential surface 410g of the first cover 410 or both of the first cover 410 and the second cover 320′ may be prevented from separating by being bonded.

Accordingly, the first cover 410 and the second cover 320′ may be integrally formed as the distal end cover 501.

The distal end of the sheath 115 is water-tightly fixed to an outer circumferential surface 320g of the second cover 320′ by, for example, bonding and fixing or sandwiching.

Note that the sheath 115 may be water-tightly fixed to the outer circumferential surface 410g of the first cover 410 or may be water-tightly fixed to the respective inner circumferential surfaces 320n and 410n. A method of causing the sheath 115 to adhere to the outer surface of the insertion section 55 and a configuration of the sheath 115 are the same as the method and the configuration in the first embodiment explained above.

The light transmitting plate 490 is formed from a material having light transmissivity and is water-tightly attached to the first cover 410 to close the opening 419 of the first cover 410 as shown in FIG. 23. Note that the light transmitting plate 490 may be water-tightly attached to the second cover 320.

The light transmitting plate 490 includes a part located in the visual field range of the observation window 352 by closing the opening 419 of the first cover 410 after the attachment.

Further, the light transmitting plate 490 includes a first surface 490a and a second surface 490h. The first surface 490a is a surface opposed to the observation window 352 when the light transmitting plate 490 is attached to the first cover 410. The second surface 490b is a surface located on an opposite side (rear surface side) of the first surface 490a. The second surface 490b is sprayed with the fluid from the nozzle 312 and includes a part located in the visual field range of the observation window 352 when the light transmitting plate 490 is attached to the first cover 410.

A flow channel connecting section 312′ is inserted into the first cover 410 via the through-hole 410p as indicated by an alternate long and short dash line in FIG. 25 when the inner circumferential surface 320n of the second cover 320 is engaged with the outer circumferential surface 410g of the first cover 410 to adhere thereto as shown in FIG. 25. As a result, the flow channel connecting section 312′ is located at a proximal end of the opening 419.

Note that when the second cover 320′ is engaged with the outer circumferential surface 410g of the first cover 410, the flow channel connecting section 312 adheres to, with an elastic force, the inner circumferential surface 410n of the first cover 410 and both the surfaces of the distal end side inclined section 362s (see FIG. 21) in a watertight state.

In this state, when the distal end cover 501 is attached to the outer circumferential surface 351g of the distal end portion 351 as shown in FIG. 23, the flow channel connecting section 312′ closes the upper part of the distal end side inclined section 362s of the fluid passage 362 exposed at the distal end portion 351. Therefore, the distal end opening 362i facing the light transmitting plate 490 at a proximal end of the opening 419 is formed as the discharge port 312t of the fluid passage 362 by the flow channel connecting section 312. The fluid passage 362, the upper part of which is closed by the flow channel connecting section 312′, configures the flow channel 321. In other words, the flow channel connecting section 312′ configures a nozzle opposed to the observation window 352.

The nozzle 312′ is a nozzle for spraying fluid from the discharge port 312t′ to a part located in the visual field range of the observation window 352 on the second. surface 490b of the light transmitting plate 490.

Note that, as explained above, the first cover 410 is formed from the hard resin in order to prevent deformation due to pressure at the time when the fluid is sprayed from the nozzle 312′.

The insertion section 55 is inserted into the body cavity in a state in which the distal end cover 501 is attached to the distal end portion 351 and the sheath 115 is caused to adhere to the outer surface of the insertion section 55 as explained above.

Thereafter, when a part located in the visual field range of the observation window 352 is dirty and observation is difficult on the second surface 490b of the light transmitting plate 490, the gas feeding and liquid feeding switching button 56b is operated by the operator, whereby fluid is supplied from the discharge port 312t of the nozzle 312 to the second surface 490b, the contaminants are removed, and a visual field of the observation window 352 is secured.

Note that although the distal end cover 501 is described as detachably attachable to the outer circumferential surface 351g of the distal end portion 351, actually, when the distal end cover 501 is detached from the distal end portion 351 after use, the distal end cover 501 is detached from the distal end portion 351 by being broken. Thereafter, the distal end cover 501 is discarded and cleaning, disinfection, and sterilization treatment is applied to the entire endoscope 50.

As explained above, an object is to provide the distal end covers for endoscope 301 and 501 and the endoscope apparatus 100 in which the fluid supply nozzle 312 or 312′ for removing, with the fluid supplied from the fluid passage 362, a target object disturbing the visual field of the observation window 352 of the endoscope 50 can be attached to the insertion section 55 of the one side view-type endoscope 50 usable in both cases of a form of use in which the insertion section 55 is covered by the sheath 115 and a form of use in which the insertion section 55 is not covered by the sheath 115 and including the fluid passage 362.

Note that, in the present embodiment as well, in a configuration in which the opening 419 is closed by the light transmitting plate 490, the same effects as the effects of the modification of the first embodiment explained above can be obtained even if the external channel 200 extended in the longitudinal axial direction N outside the insertion section 55 is fixed to, for example, the first cover 410 via the coupling member 210 having flexibility as shown in FIG. 19 referred to above.

Further, it goes without saying that, as in the first embodiment, a light transmitting plate in which an opening is formed in a position opposed to the treatment instrument raising base 390 may be provided in the opening 319 of the first cover 310 of the distal end cover 301 as well.

What is clamed is:

1. A distal end cover for endoscope attached to and detached from a distal end portion of an insertion section of an endoscope, the distal end cover for endoscope comprising:
a first cover including a nozzle for spraying fluid to at least an observation window provided at the distal end portion of the insertion section or a part located in a visual field range of the observation window; and
a second cover, at least a part of which engages with a circumferential surface of the first cover, the second cover including a flow channel for circulating, to the nozzle, the fluid fed from a fluid passage of the endoscope.

2. The distal end cover for endoscope according to claim 1, wherein
the second cover is formed from a member having more elasticity than the first cover, and
when the first cover and the second cover engage, the flow channel circulates the fluid to the nozzle in a watertight state for the nozzle with an elastic force of the second cover.

3. The distal end cover for endoscope according to claim 2, wherein
a plurality of kinds of the first cover are present, and
the second cover has a structure that engages with all of the plurality of kinds of the first cover.

4. The distal end cover for endoscope according to claim 2, further comprising a sheath water-tightly attached to an outer circumferential surface or an inner circumferential surface of the first cover or the second cover and covering an outer surface of the insertion section of the endoscope in a longitudinal axial direction of the insertion section.

5. The distal end cover for endoscope according to claim 2, further comprising a light transmitting plate having light transmissivity including a part water-tightly attached to the first cover or the second cover and located in the visual field range of the observation window and including a first surface opposed to the observation window of the insertion section and a second surface located on an opposite side of the first surface and sprayed with the fluid from the nozzle.

6. The distal end cover for endoscope according to claim 2, wherein the second cover has a tubular shape in which, when the second cover is attached to an outer circumferential surface of the distal end portion of the insertion section with the first cover, a proximal end side portion of the second cover adheres to the outer circumferential surface of the distal end portion and an outer circumferential surface of the first cover.

7. The distal end cover for endoscope according to claim 2, wherein a part of an inner circumferential surface of the second cover adheres to an outer circumferential surface of the first cover.

8. The distal end cover for endoscope according to claim 7, wherein
a through-hole passing through the outer circumferential surface and an inner circumferential surface of the first cover is formed in the first cover,
the second cover includes a flow channel connecting section projecting in a radial direction from the inner circumferential surface of the second cover, and
when the outer circumferential surface of the first cover and the inner circumferential surface of the second cover engage, the flow channel connecting section is inserted into the first cover via the through-hole.

9. The distal end cover for endoscope according to claim 8, wherein, when the second cover is engaged with the outer circumferential surface of the first cover, the flow channel is connected to the nozzle in a watertight state by an elastic force of the flow channel connecting section of the second cover.

10. The distal end cover for endoscope according to claim 9, wherein, when the first cover is attached to the distal end portion of the insertion section of the endoscope, the flow channel is connected to the fluid passage of the endoscope in a watertight state with an elastic force of the flow channel connecting section of the second cover.

11. The distal end cover for endoscope according to claim 1, wherein
the first cover includes, on an inner circumferential surface, a second locking section that engages with a first locking section provided on an outer circumferential surface of the distal end portion of the insertion section, and
the first locking section and the second locking section engage, such that positioning of the distal end portion and the first cover is performed.

12. The distal end cover for endoscope according to claim 1, wherein a channel extending along the insertion section outside the insertion section of the endoscope is attached to the first cover.

13. The distal end cover for endoscope according to claim 12, wherein the channel is provided separately from the flow channel provided in the second cover.

14. The distal end cover for endoscope according to claim 12, wherein the channel is attached to the first cover via a coupling member having flexibility.

15. The distal end cover for endoscope according to claim 12, wherein the flow channel provided in the second cover is caused to communicate with the fluid passage of the endoscope.

16. An endoscope apparatus comprising:
an endoscope including an insertion section inserted into a subject;
a distal end cover for endoscope attached to and detached from a distal end portion of the insertion section, the distal end cover for endoscope including a first cover including a nozzle for spraying fluid to at least an observation window provided at the distal end portion of the insertion section or a part located in a visual field range of the observation window and a second cover, at least a part of which engages with a circumferential surface of the first cover, the second cover including a flow channel for circulating, to the nozzle, the fluid fed from a fluid passage of the endoscope;
a sheath water-tightly attached to an outer circumferential surface or an inner circumferential surface of the first cover or the second cover and covering an outer surface of the insertion section of the endoscope in a longitudinal axial direction of the insertion section; and
a light transmitting plate having light transmissivity including a part water-tightly attached to the first cover or the second cover and located in the visual field range of the observation window and including a first surface opposed to the observation window of the insertion section to which the distal end cover for endoscope is attached and a second surface located on an opposite side of the first surface and sprayed with the fluid from the nozzle.
